(19) United States
(12) Patent Application Publication
(10) Pub. No.: US 2007/0149878 A1
(43) Pub. Date: Jun. 28, 2007

(54) APPARATUS AND METHOD FOR GUIDING A MEDICAL DEVICE IN MULTIPLE PLANES

(76) Inventor: Carol A. Hankins, Longboat Key, FL (US)

Correspondence Address: STEPTOE & JOHNSON LLP, 1330 CONNECTICUT AVENUE, N.W., WASHINGTON, DC, 20036, US

(21) Appl. No.: 11/427,070

(22) Filed: Jun. 28, 2006

Related U.S. Application Data
(63) Continuation of application No. PCT/US04/43796, filed on Dec. 28, 2004.
(60) Provisional application No. 60/532,631, filed on Dec. 29, 2003.

(51) Int. Cl. A61B 5/05

(52) U.S. Cl. 600/427

(57) ABSTRACT

The present invention provides a device for guiding a medical instrument with respect to a patient. The device includes a base. The lower portion of the base includes a bottom surface having a configuration for contact with the patient. On example of such a configuration is a generally hemispherical configuration. The device also includes an instrument guide attached to the lower portion of the base. The guide has a translation axis and is configured to slideably receive the medical instrument along the translation axis. The device further includes first and second arcuate members attached to the base. The arcuate members are generally perpendicular to each other. Moreover, the device includes first and second orientation indicators connected with the first and second arcuate members.

### CROSS-REFERENCE TO RELATED APPLICATIONS

This application is a continuation of the U.S. National Stage designation of co-pending International Patent Application PCT/US2004/043796 filed Dec. 28, 2004, which claims priority to U.S. Provisional Application No. 60/532,631 filed Dec. 29, 2003 and U.S. patent application Ser. No. 10/901,719 filed Jul. 29, 2004, and the entire contents of these applications are expressly incorporated herein by reference thereto.

### FIELD OF THE INVENTION

The present invention relates generally to an apparatus and method for guiding a medical instrument to a preselected point within a patient's body, and more particularly, to a guidance device and method of using the guidance device for inserting biopsy needles, drainage catheters, trocars, and like medical implements, into a patient's body.

### BACKGROUND OF THE INVENTION

Operators generally utilize an imaging technique, such as computed tomography (CT) or magnetic resonance (MR) scan, to identify a site of interest, such as a lesion, which requires biopsy, drainage, or other treatment. An operator may determine a best plane of imaging, the area of the lesion most likely to yield a definitive pathologic diagnosis or successful treatment, the safest pathway from the skin surface to the lesion, and the optimal size of a biopsy needle, drainage catheter, trocar, or like implement. CT and MR scanners can calculate the precise angle from the vertical position in the plane of imaging and the depth from the desired skin puncture site on the skin to the lesion. Various devices have been disclosed to guide a needle, catheter, trocar, or like implement through the skin on a precise angle and to a proper depth.

One system has been developed for CT scanners by Philips Medical Systems and is called the Pinpoint® System. This system includes a stereotactic arm with a laser beam, integrated software, and optional needle/catheter guide attached in front of the CT gantry. The laser beam indicates the desired plane of imaging, angle from vertical position, and depth.

Another system has been developed for CT and MR scanners by Ultraguide, called CT-Guide® and MR-Guide®. Electromagnetic sensors are placed on the patient and the needle or catheter outside of the CT gantry for real-time image-based tracking of the needle or catheter.

A disposable device for CT-guided procedures has been developed by Inrad, Inc., called the AccuPlace®—Drace Stereotaxic Needle Guide. The goniometer includes two rings assembled such that their radial axes are in one plane. The outer ring includes a fixed bubble level. The inner ring rotates to the desired angle within the outer ring and is bisected by a carrier for variable needle guide inserts. The needle guide inserts consist of upper and lower struts. The diameter of the goniometer is slightly greater than the length of a conventional instrument guide. In use, the device is aligned with the image plane. The axis of force to stabilize the device on the patient is different than the axis of force to advance the instrument into the patient. Relatively long segments of the instrument between the device and the patient may be unsupported by the needle guide.

Also, fluoroscopy systems are available for new CT scanners, allowing for realtime tracking of the needle, catheter, or like implement inside of the CT gantry. However, performing a procedure inside the CT gantry is awkward and occasionally not possible due to space constraints. Furthermore, the patient is exposed to additional radiation during is procedure. The operator's hands are also exposed to additional radiation, despite the use of thin collimation of the CT beam, lead plates and extended needle holders (called Instant Intervention devices by Hakko Shoji Company).

U.S. Pat. No. 4,733,661 issued to Palestrant discloses a guidance device for CT guided biopsy and drainage procedures. The guidance device includes a planar base including a bubble level to aid in maintaining the base horizontal. A needle support arm is pivotally secured to the base adjacent one end thereof, and a cooperating protractor indicates the relative angular relationship between the needle support arm and the base. Needle guides are provided on the support arm for slidingly supporting the needle/catheter at a desired angle as the needle/catheter is inserted into the patient's body. Graduations are marked on the needle support arm for indicating the depth of insertion. A reference line formed upon the base is adapted to be aligned with the image plane indicted by a transverse light beam projected by the CT scanner.

U.S. Pat. No. 4,883,053 issued to Simon discloses a self-supporting angulator device for precise percutaneous insertion of a needle or other object. The device is erectable, self-supporting, and collapsible. The device is composed of flexible materials which are presterilized, disposable, and directly attachable to the skin of the subject using adhesives. The angulator device includes a base plate with a semi-oval shaped aperture. Primary and secondary arched members are hingedly attached to the base plate. A coupling bracket with a needle holder is slid over the surface of the primary and secondary arched members. Moving the arched members allows the needle holder to change position and a different angle of intersection is achieved.

Also, U.S. Pat. No. 5,102,391 issued to Palestrant discloses a guidance device for CT guided biopsy and drainage procedures. The device includes a needle or catheter support to which the needle or catheter is releasably fastened. A pendulum pivotally depends from a pivot point on the catheter support under the force of gravity. A protractor is secured to either the catheter support or the depending pendulum to indicate the relative angular relationship between the needle/catheter and the pendulum.

U.S. Pat. No. 5,196,019 issued to Davis et al. discloses a goniometer for needle placement in connection with a computer tomography to direct a needle at a proper angle to reach an area for biopsy or other surgical procedures in the human body. The goniometer includes a double ring. The outer ring has arcuate graduations and a level mounted thereon. The inner ring is rotatable in the outer ring to various angle positions and includes a needle carrier for receiving a needle holder. The outer ring has serrations on a continuous arcuate surface and the inner ring has resilient detents to contact and ride on the serrations. A lever operated needle holder has a cam to lock a detent into a serration when the needle holder is moved to a position to retain a needle.

Finally, U.S. Pat. No. 5,314,432 issued to Paul discloses a lumbar spinal disc trocar placement device. The device includes a trocar support pivotally secured to a base plate. Bubble gauges are employed to level the x-axis and y-axis of a trocar support, which orients and slidably guides the medical probe into the targeted herniated disc nucleus.

These products are either expensive, high maintenance, time-consuming to set up, difficult to use on curved and lateral surfaces, require a conventional needle guide, or difficult to hold manually or mechanically. Consequently, these products have very low rates of clinical acceptance, and the vast majority of operators continue to use free-hand technique for initial placement and repositioning of a needle, catheter, trocar, or like implement. This entails free-hand approximation of the desired plane of imaging, free-hand approximation of the desired angle from vertical position, and free-hand approximation of the desired depth. A very limited scan is then done to see how close the medical implement is to the lesion. To reposition the medical implement, free-hand technique is used again. These steps may be repeated multiple times to achieve the desired result, depending on the technical difficulty of the diagnostic or therapeutic procedure and the number of biopsy specimens, drainage catheters, or therapies needed.

Therefore, there exists a need for an inexpensive, practical, and versatile device to facilitate accurate positioning of a medical implement in the desired plane of imaging or outside a standard plane of imaging, at the desired angle from vertical position, and to the desired depth for CT or MR-guided procedures. More accurate initial placement would reduce or eliminate the need for repositioning and would reduce the procedure time, discomfort, and risk for the patient. Ideally, this device should be easy to sterilize and/or disposable and useful on any surface of the body. It may be paired with one of the commercially available needle/catheter guides that can accommodate various sizes of medical implements, allow for resistance-free advance of the implement, and allow for quick release and recapture of the implement during the procedure. Alternatively, an instrument guide may be integral, or even of unitary construction, with the positioning device.

### SUMMARY OF THE INVENTION

One aspect of the invention relates to a positioning device that maybe used to guide an instrument and/or medical implement. For example, the positioning device may be used to guide the instrument along a target axis with respect to a patient. An exemplary target axis is the path along which an operator seeks to guide the instrument toward a target site within a patient. For example, the target axis may include a pathway between the skin of a patient and a lesion within the patient. The orientation of the target axis may be determined on the basis of imaging techniques such as x-ray and magnetic imaging techniques. Once an instrument has been guided to a desired location and/or orientation, an operator may perform a procedure. Exemplary procedures include, but are not limited to, biopsy, aspiration, drainage, chemical ablation, radiofrequency ablation, microwave ablation, laser ablation, and cryoablation.

A preferred medical implement is an elongate instrument having a longitudinal axis. A suitable instrument may include at least one of a needle, a catheter, a trocar, a cannula, an electrode, a cryoprobe and the like. The term implement also includes instruments referred to as applicators.

The type of instrument or applicator used with the present invention depends upon the type of image-guided procedure being performed. In accordance with established standards within the medical community, a needle may be utilized during a biopsy, aspiration, and chemical ablation while a catheter may be used in a drainage procedure as well as chemical ablation. During a radiofrequency ablation, an operator may use electrodes and/or cluster electrodes with the present invention. Procedures such as cryoablation, microwave ablation, and laser ablation may be performed using a cryoprobe, antennae, and fiber, respectively.

The positioning device may include a handle, an instrument guide, and an orientation device The positioning device of the present invention may be presterilized, preferably, but not necessarily, by the manufacturer. The handle preferably allows a user to manipulate the device using either hand or both hands. The handle may be modified to allow the device to be stabilized and/or manipulated by a mechanical arm. The instrument guide is preferably configured to allow a user to translate an instrument along a translation axis with respect to the positioning device. The instrument guide may be configured to prevent rotation of the instrument with respect to the instrument guide in at least one dimension. In one embodiment, suitable instrument guides include capture and release mechanisms, such as those useful for slender instruments having a longitudinal axis, including a needle, a catheter, trocar, and like implement. In another embodiment, the instrument guide is configured to contact the instrument at at least two locations spaced apart along the longitudinal axis of the instrument. For example, such an instrument guide may include one or more of a groove, a slot, a channel, and a yoke.

The instrument guide is preferably securable with respect to the handle. For example, the instrument guide preferably may be secured in use with respect to the handle, such as to prevent rotation of the instrument guide with respect to the handle when guiding the instrument along the translation axis. In one embodiment, the handle and instrument guide may be integral or even unitary with one another, like an integrated groove. In another embodiment, the integrated groove may be configured for attachment of an external or conventional instrument guide. The external instrument guide may be used for small implements. The external guide may be removed, and the integrated groove may be used to guide larger implements.

The positioning device may also include an orientation indicator, which may provide orientation data indicative of an orientation of the orientation indicator. In some embodiments, the orientation data may be deviation data, which are preferably indicative of a deviation between the orientation indicator and a reference, such as at least one of a reference point, reference axis, and reference plane. Alternatively, or additionally, the orientation data may be indicative of a deviation between the translation axis and the target axis, such as a deviation between the translation axis and the target axis within at least one plane containing the target axis. In one embodiment, the at least one plane includes the target axis and a reference axis. The reference axis may be a vertical axis. The orientation data may also, or alternatively, be indicative of a deviation between the orientation indicator and a plane containing the target axis and a reference axis, which may be a vertical axis.

At least one of the orientation indicator and handle is preferably movable with respect to the other. Rotation is a preferred movement. For example, the orientation indicator may be rotatable with respect to the handle. The device may include a reversible arcuate portion defining an arcuate path along which the orientation indicator may rotate with respect to the handle.

Preferably, the orientation indicator and handle are stably positionable with respect to one another. By stably positionable it is meant that, upon movement with respect to one another to a stable relative position, the relative orientation between the handle and the orientation indicator resists change unless desired by an operator For example, the positioning device may be provided with an amount of friction between the orientation indicator and handle that is sufficient to resist movement unless manipulated by an operator. Alternatively, or in combination with frictional resistance, the orientation indicator and handle may be movable to a plurality of positions defined by detents. A tension adjustment device such as a screw may be provided to secure the relative positions of the orientation indicator and handle. Of course, combinations of such structure and properties may be used to provide stable positioning of the orientation indicator and handle yet allow relative movement.

A preferred orientation indicator is sensitive to gravity. One example of a suitable orientation indicator is a trapped fluid or trapped bubble indicator, which may provide orientation data in the form of a position of a fluid or bubble within the indicator. Another suitable indicator includes a movable element, such as a ball bearing, movable with respect to an arcuate surface, such as a convex dish-like surface.

In one embodiment, the positioning device is a disposable, single use device that may be manufactured inexpensively and discarded after use. In another embodiment, the positioning device may be sterilizable for repeated use. Whether or not intended as a disposable device, the handle may be of integral, even unitary construction. For example, the handle may be formed of a polymer, which may be molded as by injection molding or other technique. In embodiments including an arcuate portion, the arcuate portion may be of integral or unitary construction. The arcuate portion may be of integral or unitary construction with respect to the handle. Alternatively, the arcuate portion may be positionable in at least two orientations with respect to the handle. A positioning device of the invention may be sterilized, for example during manufacture. In this embodiment, the positioning device may be shipped within a sterile enclosure to another location for use in a procedure.

Another aspect of the invention relates to a method for guiding an instrument along a target axis, such as a target axis within a patient. The method may comprise providing a positioning device, which may include a handle, an instrument guide, and an orientation indicator. The instrument guide allows an operator to guide an instrument along a translation axis, which is preferably aligned with the target axis during the procedure. The instrument guide is preferably secured in use with respect to the handle.

The orientation indicator preferably provides deviation data indicative of a deviation between the orientation indicator and a reference axis within at least a first plane containing the reference axis. For example, the reference axis may be a vertical axis.

The method further comprises rotating at least one of the orientation indicator and the handle with respect to the other into a stable relative orientation of the orientation indicator and the translation axis. The deviation data is observed. For example, if a trapped fluid orientation indicator is used, the operator may observe a relative position of the trapped fluid. The handle is rotated, preferably with respect to the patient, to seek a predetermined deviation between the orientation indicator and the reference axis within the first plane. For example, one may rotate the handle to reduce the deviation where a minimal deviation between the orientation indicator and reference axis is sought.

A further aspect of the present invention relates to another embodiment of a medical implement positioning device. The lower portion of the base of the device includes a bottom surface having a configuration for contact with a patient. The bottom surface may be configured for rotation in multiple planes (e.g. a generally hemispherical configuration). The device includes an instrument guide attached to the lower portion of the base. The guide has a translation axis and is configured to slideably receive the medical implement along the translation axis. The positioning device also includes first and second arcuate members attached to the base. The arcuate members are generally perpendicular to each other. Finally, the device includes first and second orientation indicators connected with the first and second arcuate members, respectively.

In a related aspect, the positioning device may be used for guiding a medical instrument along a target axis within a patient. The method includes positioning the medical instrument in the instrument guide, positioning the bottom surface of the base against the patient with the distal end of the medical instrument positioned near a puncture site of the patient, rotating the positioning device about the bottom surface until the translation axis is generally aligned with the target axis, and translating the medical instrument along the translation axis and through the puncture site of the patient.

The method may further include determining first and second deviation angles of the target axis in first and second planes. The first orientation indicator is positioned on the first arcuate member at indicia representing the first deviation angle, and the second orientation indicator is positioned on the second arcuate member at indicia representing the second deviation angle. Also, the first arcuate member is positioned generally parallel with the first plane, and the second arcuate member is positioned generally parallel with the second plane to thereby align the translation axis with the target axis.

The present invention has many advantageous features. The device can be significantly larger than a conventional instrument guide, without interfering with the use of a manual, semi-automated, or automated biopsy device. It can have a significantly larger angle indicator with easy to read numbers. It has an ergonomic handle, which can have height/depth indicators and be modified for use with a mechanical or robotic arm. The instrument guide is always directly on or close to the skin. Therefore, the distal end of the instrument is always stabilized by the instrument guide and is less likely to deviate from the target axis. In use, the bubble level is rotated to the desired angle of the procedure; then the entire device is rotated relative to the patient. Therefore, the axis of force to stabilize the device is the same as the axis of force to advance the instrument. Consequently, the device is easier to use, particularly on curved surfaces. Finally, one embodiment facilitates accurate positioning of a medical implant outside of a standard plane of imaging.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front view of an exemplary embodiment of the medical implement positioning device;

FIG. 2 is a side view of the exemplary embodiment;

FIG. 3 is a top view of the exemplary embodiment;

FIG. 4 is a front view of an alternative embodiment of the orientation indicator and arcuate member;

FIG. 5A is a front view of a sliding marker for the alternative embodiment of the orientation indicator and arcuate member of FIG. 4;

FIG. 5B is a top view of the sliding marker;

FIG. 6 is a side view of the positioning device showing a notch for the arcuate member and an attachment point for a mechanical arm;

FIG. 7 is a front view of another alternative embodiment of the orientation indicator and arcuate member;

FIG. 8 is a front view of a simplified instrument guide;

FIG. 9 is a top view of the simplified instrument guide;

FIG. 10 is a top view of one embodiment of the simplified instrument guide;

FIG. 11 is a top view of another embodiment of the simplified instrument guide;

FIG. 12 is a side view of one embodiment of the simplified instrument guide;

FIG. 13 is a side view of another embodiment of the simplified instrument guide;

FIG. 14 illustrates exemplary orientations of the present invention;

FIG. 14A is an expanded view of FIG. 14;

FIG. 15 is a side view of an ultrasound transducer with a simplified instrument guide;

FIG. 16 is a top view of the ultrasound transducer covered in a sterile sleeve;

FIG. 17 is a top view of the ultrasound transducer covered in a sterile sleeve having a V-shaped groove placed adjacent the simplified instrument guide;

FIG. 18 is a front view of another exemplary embodiment of the medical implement positioning device;

FIG. 19 is a side view of the positioning device of FIG. 18; and

FIG. 20 is a top view of the positioning device of FIG. 18.

### DETAILED DESCRIPTION OF THE INVENTION

The present invention relates to a device and method for allowing an operator to introduce an instrument or medical implement to a site within a patient. The positioning device of the invention may be designed for use with the operator standing along either side and facing the long axis of a support, such as a table, of an imaging system and using either hand to hold the device. For example, the operator may wish to biopsy a site, drain a site, treat a site, and/or implant a catheter or other device. In such methods, the operator may use image data, which may generally be acquired prior to the procedure, to choose at least one of an optimal imaging plane, an optimal area of the lesion for biopsy/drainage, the approximate biopsy/drainage pathway, the approximate skin puncture site, and most appropriate instrument for the procedure.

Preliminary image data through the general area of the site, such as CT or MR data, may be used, for example, to choose the plane and pathway for the procedure. The preliminary image data may be acquired generally concurrently with the procedure using preliminary skin markers. On a preliminary image, the operator may place a cursor on the desired area of the lesion for biopsy/drainage and the desired skin puncture site. Software of the imaging device may depict the desired biopsy/drainage pathway on the image and calculate its orientation with respect to a reference axis, such as a vertical axis, and, optionally, the depth from the skin to the lesion. A laser beam built into the imaging system may be used to project the plane of the image onto the patient. The target axis along which the operator will introduce the instrument may occupy the image plane.

The operator may place markers on the skin in the area of the desired skin puncture site and in the plane of the image. Using sterile techniques, the skin at the desired puncture site may be anesthetized and incised. The positioning device of the present invention may then be utilized to perform the required medical procedure.

For difficult CT-guided procedures, such as when a critical structure is between the skin puncture site and lesion, the operator may choose a pathway which is not in a standard transverse vertical imaging plane. The software of the newer imaging systems can generate reconstructed images in any non-orthogonal vertical plane, which can be used in the same way to depict the desired procedure pathway on the image and calculate its orientation with respect to a reference axis, such as a vertical axis, and optionally, the depth from the skin to the lesion.

The operator may place preliminary skin markers in the approximate area of the desired skin puncture site and on the skin above the lesion to indicate the desired plane of the procedure. A scout image can depict these markers and be used for orientation of the reconstructed images. The operator may place markers on the skin in the area of the desired skin puncture site and in the plane of the reconstructed images. Using sterile techniques, the skin at the desired puncture site may be anesthetized and incised. The positioning device of the present invention may then be utilized to perform the required medical procedure.

However, for difficult CT-guided procedures, such software may not be easy to use or available for all operators. In this case, the operator may use known deviation angles to align the medical implement with the desired pathway. For example, the first deviation angle is again calculated from the standard transverse vertical imaging plane, and the second deviation angle is the known degree of tilt of the CT gantry. Since the desired pathway lies outside the standard imaging plane, the operator may move the medical implement in multiple planes to align the implement with the target axis.

It should be understood that the present invention is not limited to the aforementioned techniques. Rather, these techniques are merely discussed to provide an exemplary context in which one may use the present invention.

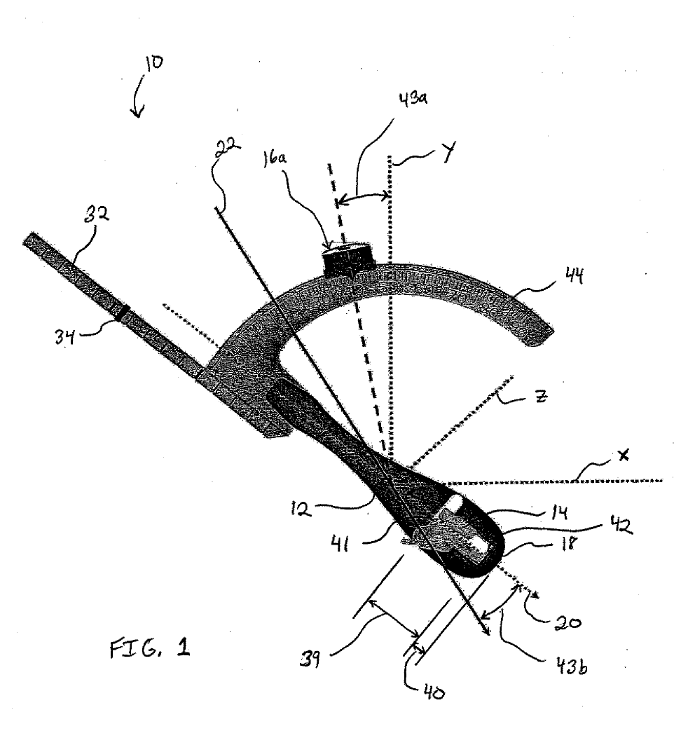

Referring to FIGS. 1-3, a positioning device 10 may include a handle 12, an instrument guide 14, and an orientation indicator 16a. The handle 12 may operate as a base of the positioning device 10. In one embodiment, the handle 12 includes a lower surface 18, which may be positioned adjacent or against a patient during use. The lower surface 18 may occupy a plane orthogonal to the image plane and be configured for rotation on the patients skin. The lower surface 18 may have a cross section that is arcuate. Lower surface 18 may have a radius of curvature of about, for example, 3 cm to 0.5 cm. Preferably, the radius of curvature is 1.5 cm. In use, an operator may contact the patient with lower surface 18 and, optionally, simultaneously rotate the device 10 with respect to the patient. Thus, in use, the positioning device 10 may include a lower surface 18 which may contact a patient and may be rotated by an operator with respect to the patient while the operator adjusts an orientation of the translation axis 20 with respect to a desired target axis 22. When placed in contact with the patient, the lower surface 18 may sink into and be stabilized by the soft tissues with the distal portion 42 of the instrument guide 14 positioned against but not significantly depressing the surface of the skin. This allows the device to be stabilized without altering the calculated depth of penetration.

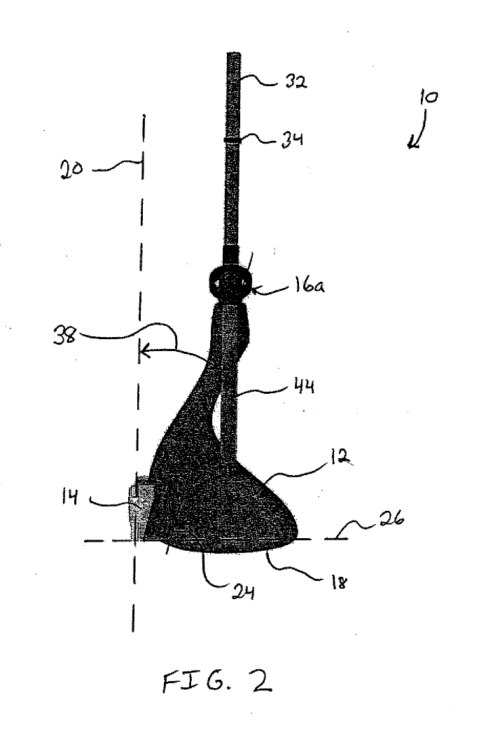

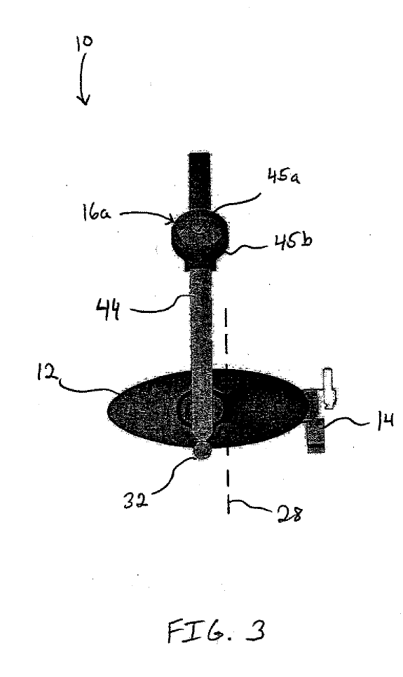

As shown in FIGS. 2 and 3, the handle 12 may include a major axis 24, a horizontal axis 26, and a minor axis 28. The major axis 24 may be in the same plane as the translation axis 20 of an instrument with respect to instrument guide 14. In use, the horizontal axis 26 may be oriented perpendicular to an imaging plane. Because a dimension of handle 12 along horizontal axis 26 is preferably greater than a dimension of handle 12 along minor axis 28, lower surface 18 helps stabilize the positioning device 10 with respect to motion perpendicular to the image plane. For example, a maximum dimension of handle 12 along minor axis 28 may be about 6 cm or less, e.g., about 3 cm or less. A maximum dimension of handle 12 along horizontal axis 26 may be about 12 cm or less, e.g., about 8 cm or less, such as 7 cm or less. The handle 12 may be configured to allow the right or left hand of an operator to freely and manually manipulate the device 10.

The handle 12 may include markers, such as vertical markers, allowing a user to determine a distance which an instrument has translated with respect to the handle 12. The markers may be centimeter markers on the front and sides and may include a bold score at desired intervals (e.g., every 5 or 10 cm). The first centimeter marker is preferably at the level of the distal portion 42 of the instrument guide 14. The positioning device 10 may include a vertical ruler 32, fixed or detachable with respect to the handle 12 and parallel to the translation axis 20 of an instrument operatively associated with the instrument guide 14. A slide 34 may be used to mark the distance of the desired penetration depth from the level of any reference point on the instrument.

As further seen in FIGS. 1-3, the instrument guide 14 preferably allows the operator to translate an instrument along a translation axis 20 of the instrument guide 14. The instrument guide 14 may be a commercially available instrument guide, such as a needle/catheter guide or a built-in needle/catheter guide. The instrument guide 14 may be secured, either permanently or releasably, with respect to the handle 12, such as to a lateral portion thereof The lower portion of the handle 12 may be configured for receiving the instrument guide 14 by any known means, including, for example, by way of clip-on, slide-on, fastening, etc. Preferably, instrument guide 14 holds or guides the instrument such that the instrument may translate along the translation axis 20 of the guide 14 but is generally limited from moving away the translation axis 20. The translation axis 20 of the guide 14 may be substantially parallel with or at an offset angle to the major axis 24 of the handle 12. For example, an angular offset 38 between the major axis 24 of the handle 12 and the translation axis 20 may be less than 45 degrees, for example less than around 35 degrees during use. The instrument guide 14 may include side markers to align with the plane of imaging and the skin puncture site.

Instrument guide 14 and handle 12 may be configured to allow use of a mechanical device for manipulating the instrument. Exemplary mechanical devices include biopsy guns, such as the Biopty® device. Another suitable device is an automated, spring-propelled dual stage tissue sampling device as described in U.S. Pat. No. 4,699,154 to Lindgren et al., which is hereby incorporated by reference. Preferably, instrument guide 14 holds or guides a distal portion of the instrument which allows a proximal portion of the instrument to be received by the mechanical device. Translation axis 20 of the guide 14 and the major axis 24 of the handle 12 may be offset from one another by the offset angle 38, thereby creating a distance therebetween of about 1 cm to 7 cm. In an exemplary embodiment, the distance between the translation axis 20 and the major axis 24 formed by the offset angle 38 is at least 4 cm.

The instrument guide 14 may have a dimension 39 along the translation axis 20 of about 1 cm to 8 cm between a proximal portion 41 and distal portion 42 of the instrument guide 14. A dimension 40 between lower surface 18 and the distal portion 42 of instrument guide 14 may be about 1 cm or less. Dimension 40 may be the distance that the lower surface 18 sinks into the patient's skin allowing the distal portion 42 of the instrument guide 14 to rest against, or at least be closely positioned to, the patient's skin.

After initial placement of the instrument, a quick release mechanism of the instrument guide 14, if used, may be used to remove the positioning device 10. A limited scan may be obtained to assess positioning of the instrument. If adjustment of the position of the instrument is desired, the quick recapture mechanism of the instrument guide 14 may be used to reposition the positioning device 10.

The positioning device 10 may include an orientation indicator 16a, such as a gravitational indicator, which may be a trapped fluid and gas indicator. In addition to the orientation indicator 16a, the device 10 may include an arcuate member 44, such as an arc. The arc 44 may be a calibrated in degrees, for example, a 90 degree arc. The arcuate member 44 may be reversibly secured with respect to the handle 12. Preferably, in use, the arcuate member 44 is orientated generally parallel with the plane of imaging. The orientation indicator 16a of the positioning device 10 is preferably rotatable along the arcuate member 44 that may be parallel to the imaging plane. The arcuate member 44 may be secured with respect to the handle 12 such that the zero degree calibration is parallel to the translation axis 20.

The orientation indicator 1 6a may provide deviation data indicative of a deviation 43a in the orientation of the orientation indicator 16a with respect to a reference axis y. Alternatively, or in addition, the orientation indicator 16a may be indicative of a deviation 43b between the translation axis 20 and the target axis 22. Deviations 43a and 43b may both be with respect to a first plane, for example a plane containing the target axis 22 and reference axis y. The first plane may be the image plane as defined by an imaging system used to plan the procedure. The reference axis y may be a vertical axis.

Preferred orientation indicators include gravitational orientation indicators such as trapped fluid indicators including bubble in liquid, immiscible liquids. A pendulum may be used. The orientation indicator may be an element, such as a sphere, movable about an arcuate surface.

In an exemplary embodiment, the orientation indicator 16a includes a bubble 45a in a small transparent fluid-filled chamber 45b that slides along an arc 44 that is calibrated in degrees. The operator slides the center of this chamber 45b to the desired biopsy angle on the arc 44. The instrument guide 14 is loaded and adjusted. A reference point on the needle/catheter is chosen and the slide 34 on the centimeter ruler 32 is moved from this level downward to reflect the desired depth. The operator places the positioning device 10 on the patient with the tip of the needle/catheter on the skin puncture site, the side markers in the plane of imaging (indicated by the markers on the skin), and the bubble 45a centered in the chamber 45b. The positioning device 10 is now at the desired biopsy angle in the plane of imaging.

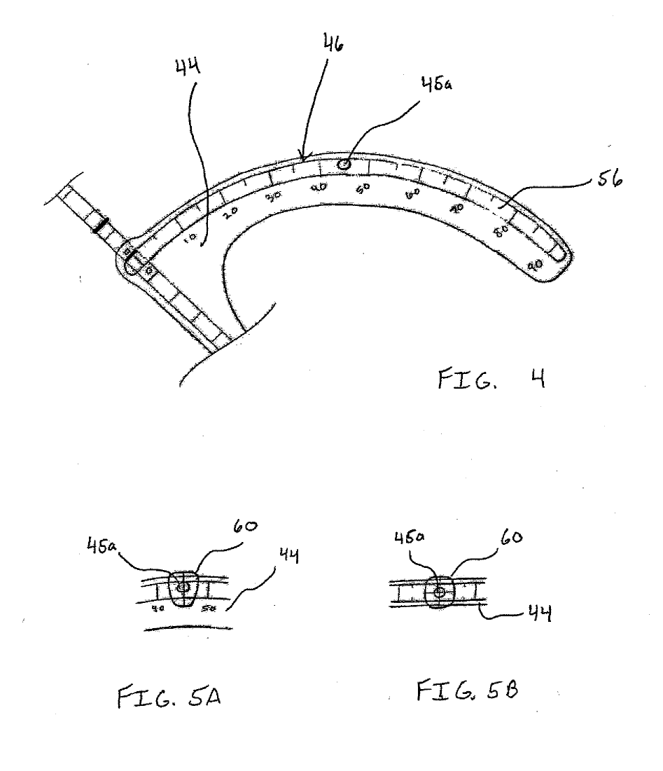

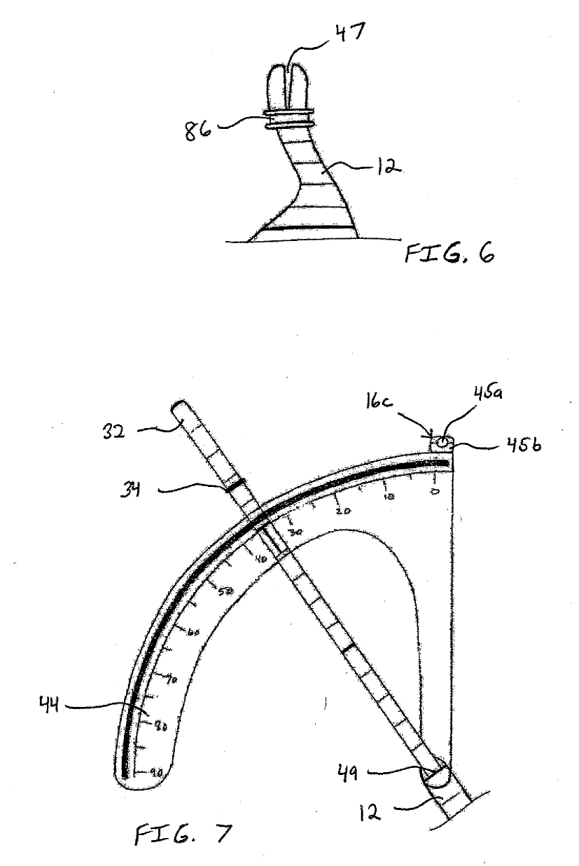

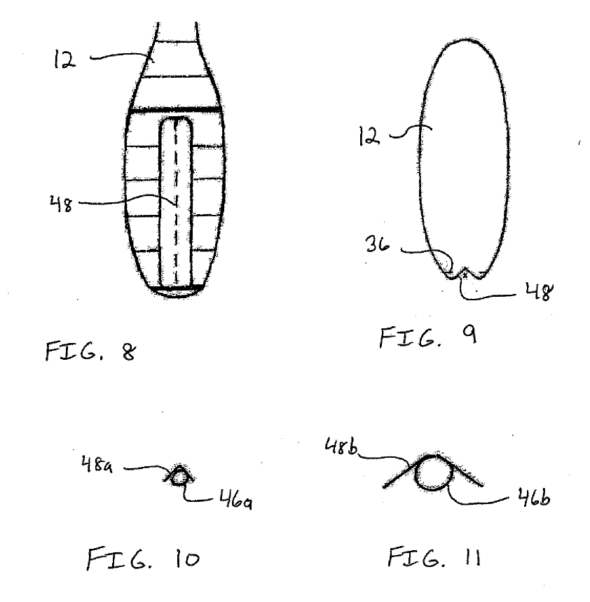

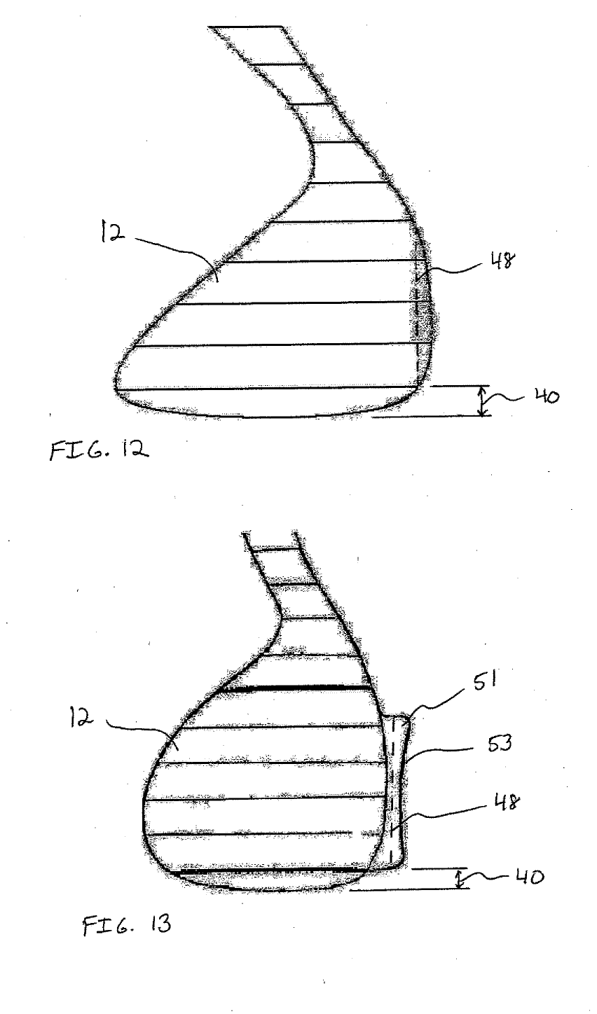

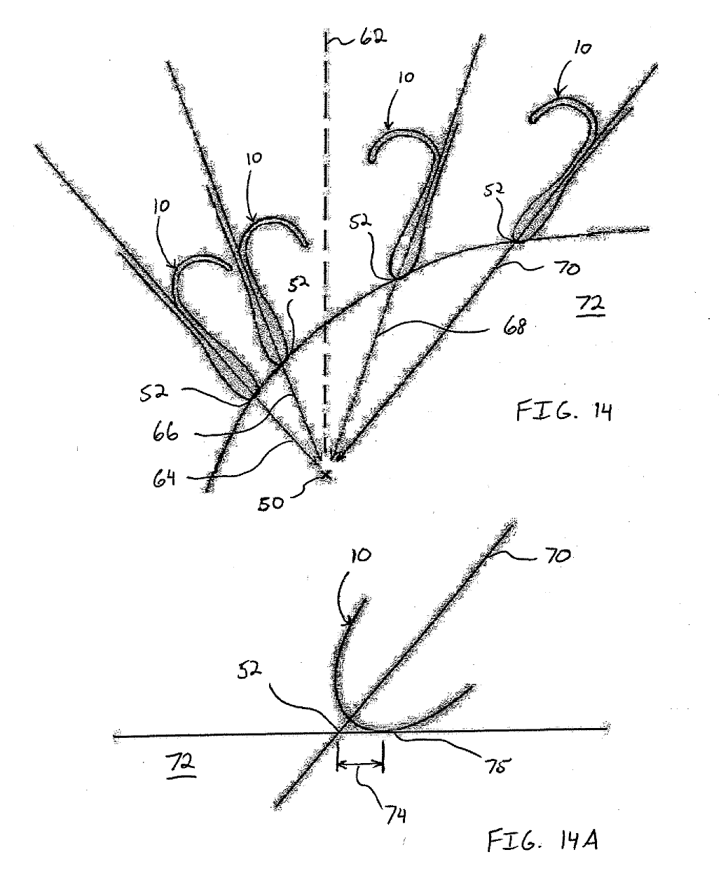

Referring to FIG. 4, an alternative orientation indicator 16b of the positioning device includes a bubble 45a in a narrow transparent fluid-filled tube 56 overlying an arc 44 that is calibrated in degrees. The arcuate member 44 may be reversibly secured with respect to the handle. The orientation indicator 16b may provide deviation data indicative of a deviation in the orientation of the orientation indicator 16b with respect to a vertical axis.

In FIGS. 5A and 5B, a sliding marker 60 is movable with respect to the arcuate member 44. The sliding marker 60 indicates a desired orientation with respect to the translation axis of the instrument guide. The sliding marker 60 may have a vertical line on the front and back and a horizontal line parallel to the arc 44 on the top. The operator moves the sliding marker 60 along the arc 44 so that the vertical line is superimposed on the desired biopsy angle. The instrument guide is loaded and adjusted. The operator places the positioning device on the patient with the tip of the needle/catheter on the skin puncture site, the side markers in the plane of imaging, and the bubble 45a centered over the vertical and horizontal lines of the sliding marker 60. The positioning device is now at the desired biopsy angle in the plane of imaging.

As previously described, the arcuate member of the positioning device may be secured to the handle. In FIG. 6, the proximal portion of the handle 12 may include a notch 47 for reversibly attaching an end portion of the arcuate member to the handle. The notch 47 may be generally parallel with the imaging plane so that when the arcuate member is inserted, the arcuate member is also generally parallel with the imaging plane.

The arcuate member 44 can be inserted into the notch 47 in different orientations. Therefore, the device can be held in either hand, used from either side of the table of the imaging system, and placed on the patient in different orientations. For example, a right-handed operator may desire to hold the device in the left hand, leaving the right hand to manipulate the medical implement. If a right-handed operator desires to perform the procedure from the right side of the table of the imaging system, the device may be placed on the patient with the instrument guide generally directed toward the patient's feet. If the procedure pathway is directed away from the operator, the reference axis is directed away from the operator, and the arcuate member is attached so that its free end is directed away from the operator (on the left side of the device). If the procedure pathway is directed toward the operator, the reference axis is directed toward the operator, and the arcuate member is attached so that its free end is directed toward the operator (on the right side of the device).

Alternatively, if a right-handed operator desires to hold the device in the left hand and perform the procedure form the left side of the table of the imaging system, the device may be placed on the patient with the instrument guide generally directed toward the patient's head. If the procedure pathway is toward the operator, the free end of the arcuate member is directed toward the operator. If the procedure pathway is directed away from the operator, the free end of the arcuate member is directed away from the operator. The device is then rotated to align the translation axis with the target axis. For a left-handed operator, the converse may be true.

The deviation angles on the arcuate member can be made readable regardless of the orientation of the arcuate member. For example, the arcuate member may be transparent so the operator can read the deviation marks from the front and back of the arcuate member.

As also shown in FIG. 6, the handle 12 includes an attachment point 86 for attaching the positioning device 10 to an attachment arm, mechanical arm, and/or robotic arm. A portion of the attachment arm may be attached to the attachment point 86 while another portion of the attachment arm may be secured to a stationary object such as, for example, the patient support table, the wall or floor of the operating/procedure room, and/or the imaging equipment. In this configuration, the positioning device 10 may be held in its desired orientation in a hands-free manner. The attachment point 86 may be located on the positioning device 10 to avoid interference with its normal functions. In an exemplary embodiment, the attachment point 86 may be located near the proximal portion of the handle 12. Preferably, the attachment arm may be joined with the attachment point 86 to form a secure and steady connection. The attachment arm may be screwed, bolted, clipped, and/or collared to the attachment point 86.

FIG. 7 illustrates another orientation indicator 16c of the positioning device. The orientation indicator 16c may include an arcuate portion 44 positioned parallel to the plane of imaging about its center with the zero degree calibration positioned vertically. The arcuate member 44 may be pivotally or reversibly attached to the handle 12. The pivot point 49 allows the arcuate member 44 to rotate about its center. The arcuate member 44 may be calibrated in degrees. A bubble 45a in a small transparent fluid-filled chamber 45b, such as a commercially available circular bubble level, may be fixed to the arcuate member 44 at 0 degrees.

In use, the operator rotates and locks the arcuate member 44 so that the center of the ruler 32 (which indicates the needle/catheter pathway of the positioning device) is superimposed on the desired biopsy angle on the arcuate member 44. The instrument guide is loaded and adjusted. A reference point on the needle/catheter is chosen and the slide 34 on the ruler 32 is moved downward to reflect the desired depth. The operator places the positioning device on the patient with the tip of the needle/catheter on the skin puncture site, the side markers in the plane of imaging, and the bubble 45a is centered within the chamber 56. The positioning device is now at the desired biopsy angle in the plane of imaging.

Like the arcuate members of orientation indicators 16a and 16b, the arcuate member of orientation indicator 16c may be reversed to accommodate various orientations of the positioning device (for example, to accommodate left and right handed operators). The arcuate member 44 may be detached from the pivot point 49 at the proximal end of the handle 12, then reversed and reattached to the pivot point 49.

It is contemplated that in addition to the orientation indicators 16a, 16b, and 16c described above, other orientations may be compatible with the present invention.

Referring to the embodiment of FIGS. 8-9, a simplified instrument guide suitable for use with the present invention may be configured to provide rapid release and/or recapture of a medical implement previously released from the instrument guide. The instrument guide may be configured to contact the medical implement at at least two points along a longitudinal axis thereof. Exemplary simplified instrument guides include at least one of a groove, yoke, a slot, and a channel 48. Preferably, the groove 48 resists movement of the medical implement in at least one, two, or at least three directions that are preferably orthogonal to the translation axis of the implement. Preferably, the groove 48 does not resist movement of the implement back and forth along at least one axis, which is preferably orthogonal to the translation axis. As further seen in FIG. 9, side markers 36 adjacent to the mid portion of the groove 48 may be included to assist with alignment of the positioning device in the plane of imaging and at the desired skin puncture site.

In one embodiment, the medical implement may be releasable from the groove 48 without mechanical manipulation thereof in at least one direction preferably orthogonal to the translation axis. The implement may additionally, or alternatively, be recapturable by the groove 48 without mechanical manipulation thereof in a second preferably different direction, which may also be orthogonal to the translation axis. An example of mechanical manipulation includes release of a spring loaded retention clip.

The medical implement may be secured with respect to the groove 48 with one or more of the operator's fingers. The use of the operator's fingers as an integral part of a simplified type of instrument guide allows for, among other things, optimal quick release and quick recapture of the medical implement, tactile control of position of the implement, and tactile control of friction during passage of the implement in the biopsy/drainage pathway.

If there is problematic friction between the sterile glove on the operator's fingers and the instrument (i.e. soft plastic drainage catheter), a preferably low friction flap, such as one comprising plastic or metal could be interposed, while maintaining stability of the instrument in the groove 48.

The groove 48 of the positioning device may be configured to align the medical implement in the translation axis and/or for placement of a range of gauge or French sizes of instruments, so that smaller instruments still can be felt with the fingertips and larger instruments are not significantly out of the plane of imaging. The angle of the V-shaped groove 48 may range from about 90 to about 170 degrees. The depth of the groove 48 may range from about 2 mm to about 10 mm. Preferably, as illustrated in FIG. 10, a groove 48a having an angle of 90 degrees and a depth of 3 mm will be used for an implement 46a with a diameter or width of 2-4 mm. Preferably, as illustrated in FIG. 11, a groove 48b having an angle of 110 degrees and a depth of 6 mm will be used for an implement 46b with a diameter or width of 5-7 mm.

In another embodiment, the groove 48 may be configured for inserts, and, optionally, detachable with replacements, to allow for variations in the angle and depth of the groove 48, with smaller angles and less depth for smaller applicators and larger angles and more depth for larger applicators, if required.

FIGS. 12 and 13 illustrate exemplary embodiments of the simplified instrument guide or groove 48. In FIG. 12, the groove 48 is integrated and recessed within the side of the handle 12. The longitudinal axis of the groove 48 generally extends orthogonal to the lower surface 18. As previously described, the dimension 40 between the distal portion of the groove 48 and the lower surface 18 is approximately 1 cm or less. The dimension 40 allows the curved or arcuate shape of the lower surface 18 to sink into the skin when the positioning device is positioned against the patient.

In FIG. 13, the groove 48 is integrated and is located in an offset portion 51 of the handle 12. The longitudinal axis of the groove 48 generally extends orthogonal to the lower surface 18. The face 53 of the offset portion 51 may be generally flat, concave or convex. Such a face 53 may improve the operator's tactile control of the medical implement positioned within the groove 48. As described above, the dimension 40 also exists in the embodiment of FIG. 13.

In further embodiments of FIGS. 12 and 13, the handle 12 or offset portion 51 may be configured to allow attachment of a conventional or external instrument guide. Preferably, the conventional instrument guide may be clipped, snapped, or fastened to the handle 12 or offset portion 51. It is contemplated that the groove 48 may be used for larger medical implements, while the attachable instrument guide may be used for smaller medical implements. For example, during a medical procedure, an operator may attach the conventional instrument guide to the handle 12 or the offset portion 51 to guide a needle or like implement then detach the instrument guide and use the V-shaped groove to guide a catheter or like implement.

Referring to FIG. 14, exemplary orientations of the positioning device 10 with respect to a reference axis 62 and target site 50 in accordance with the present are shown. Reference axis 62 may be a vertical axis, for example an axis aligned with a local gravitational field. Target site 50 may comprise, for example, a lesion that an operator wishes to biopsy. Also shown are target axes 64, 66, 68, and 70, which represent different pathways along which the operator may wish to guide an instrument toward the target site 50. Target axes 64, 66, 68, and 70 have a respective deviation of 40 degrees, 20 degrees, −20 degrees, and −40 degrees with respect to the reference axis 62. In FIG. 14, the axis of force to stabilize the device is shown to be aligned with the translation axis and the target axis. An orientation indicator of the positioning device preferably provides orientation data indicative of the alignment of the target axis and translation axis in at least one and preferably at least two dimensions. The orientation indicator may be indicative of an alignment of arcuate portion 44 with at least one plane, for example an image plane containing the target site 50.

As previously described, the positioning device 10 has a lower surface 18 which may be configured for rotation on the skin of the patient. The lower surface 18 may be arcuate. Lower surface 18 may have a radius of curvature of about, for example, 3 cm to 0.5 cm. Preferably, the radius of curvature is about 1.5 cm. As seen in FIG. 14A, when the positioning device 10 is positioned so that the target axis and instrument trajectory are aligned, a portion of lower surface 18 may contact a portion of the patient 72. The radius of curvature of about 1.5 cm on the lower surface 18 allows the lower surface 18 to contact the patient 72 relatively close to the skin puncture site 52. That is, a distance 74 between the skin puncture site 52 and the lower surface-patient contact point 75 is preferably small enough that a force directed distally along the target axis tends not to destabilize a position of the device 10 or allow deflection of the instrument from the target axis. Preferably, the distance 74 may be 3 cm or less, for example 2 cm or less.

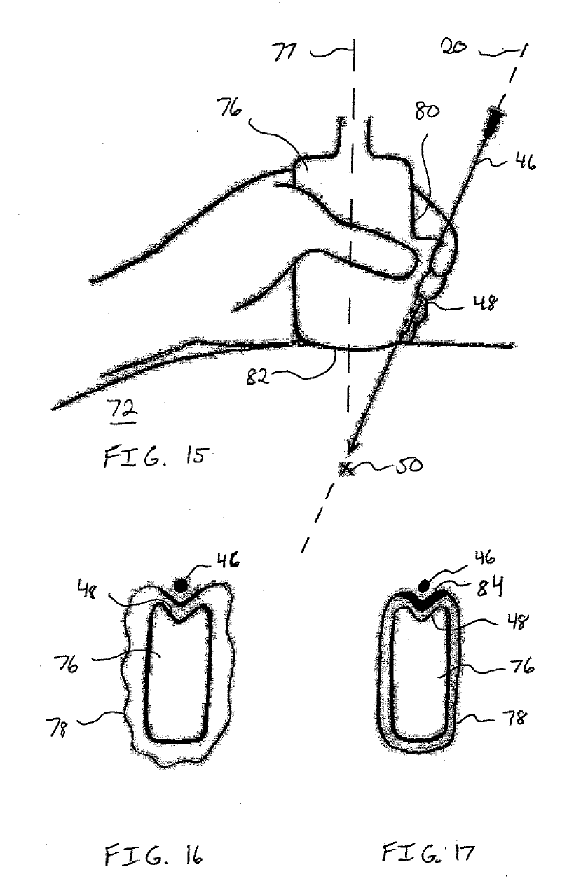

In a further embodiment of the simplified instrument guide illustrated in FIG. 15, the positioning device or the handlebase may be an ultrasound transducer 76. Currently, commercially available instrument guides for ultrasound guided procedures have the capability of quick release (upon release of a mechanical securing member), but generally not quick recapture of the instrument. For many commercially available instrument guides, low friction during passage of the instrument in the biopsy/drainage pathway is inconsistent, due to slight manufacturing variations in the true diameter of the instruments. Finally, commercially available instrument guides are attached to the ultrasound transducer by a bracket, which is relatively expensive. Therefore, there is a need for a transducer 76 with an instrument guide that is inexpensive and provides quick release and quick recapture of an instrument, applicator, or other medical implement.

A preferred medical implement 46 is an elongate instrument having a longitudinal axis. A suitable implement 46 may include at least one of a needle, a catheter, a trocar, a cannula, an electrode, a cryoprobe and like medical implements. Also, a suitable implement 46 may be configured to direct radiation, such as electromagnetic radiation, to a site of interest. Antennae, a light transmissive fiber, and microwave guides are all suitable instruments for use with the present invention.

Other medical implements previously described herein may also be utilized.

The ultrasound transducer 76 may include a simplified instrument guide which may be incorporated in the ultrasound transducer 76. A manufacturing modification may be made to the shape of the cranial side 80 of the transducer 76. A V-shaped groove 48 may extend along the cranial side 80 of the transducer 76 to the head 82 of the transducer 76, close to the skin surface of the patient 72. The groove 48 may be integrated into the cranial side of the transducer or may be offset from the transducer as described in FIG. 15. The angle from vertical position of the groove 48 should be the most commonly desired angle from vertical position for a procedure with that transducer 76.

The groove 48 allows for easy placement of the medical implement 46. The implement 46 is held in the groove 48 with the operator's fingers. Smaller instruments should be able to be felt with the fingertips; both smaller and larger instruments should be able to be firmly held within the groove 48. The use of the operator's fingers allows for optimal quick release and quick recapture of the instrument 46, tactile control of the position of the instrument 46, and tactile control of friction during passage of the instrument 46 in the biopsy/drainage pathway.

As further shown in FIG. 15, a portion of the head 82 of the ultrasound transducer 76 may occupy a plane which is generally parallel to the skin of the patient. A longitudinal axis 77 of the ultrasound transducer 76 may be generally normal to the plane. In use, an instrument 46 may be translated along a translation axis 20 of the groove 48 toward a target site 50. The translation axis 20 may extend through the plane at a zero angle with respect to the longitudinal axis 77 of the ultrasound transducer 76. Preferably, the translation axis 20 may be at a non-zero angle to the longitudinal axis 77 of the transducer 76 which is at least 10 degrees and less than 45 degrees.

Referring to FIG. 16, a customary sterile drape 78 may be placed over the transducer 76. The groove 48, covered with the drape 78, still allows for easy placement of the medical implement 46.

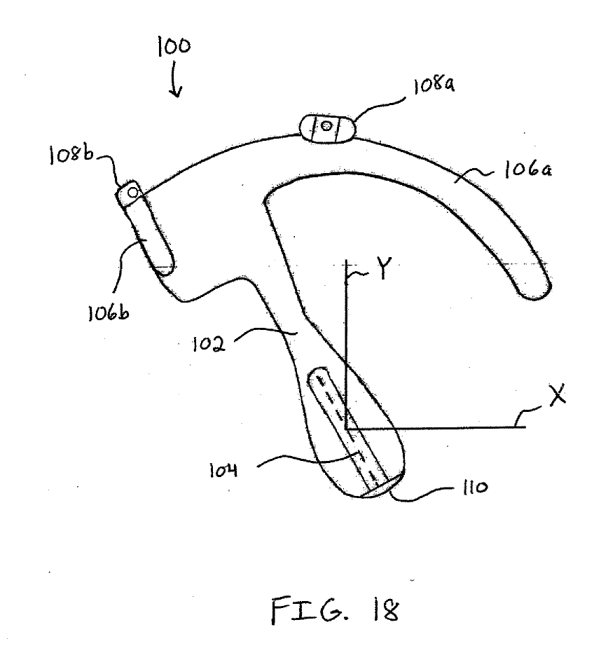

In addition, or as an alternative, a manufacturing modification may be made to the sterile sleeve 78 for the ultrasound transducer 76. As illustrated in FIG. 17, the part of the sleeve 78 covering the transducer 76 may fit more tightly and have an area of relatively hard material, such as plastic, with a V-shaped groove 84 that fits into the groove 48 of the ultrasound transducer 76. The groove 84 of the sterile sleeve 78 may allow for a range of gauge and/or French sizes of instrument. The angles and depths of the groove 84 in the sterile sleeve 78 may be similar to the groove 48 illustrated in FIGS. 10 and 11. The groove 84 of the sterile sleeve 78 may be variable, thereby functioning as an insert for the groove 48 of the ultrasound transducer 76. This would allow for variations in the cross sectional angle and depth of the groove 84, if required. Smaller angles and less depth may be needed for smaller medical implements 46; larger angles and more depth may be needed for larger implements 46.

An implement 46 placed in the groove 84 of the sterile sleeve 78 which is positioned in the groove 48 of the ultrasound transducer 76 would be in the plane of imaging and at the most commonly desired angle from vertical position for a procedure with that transducer 76. The use of a groove 84 allows for easy placement of the implement 46. The implement 46 is held in the groove 84 with the operator's fingers. Smaller implements should be able to be felt with the fingertips; both smaller and larger implements should be able to be firmly held within the groove 84. The use of the operator's fingers allows for, among other things, optimal quick release and quick recapture of the implement 46, tactile control of the position of the implement 46, and tactile control of friction during passage of the implement 46 in the biopsy/drainage pathway.

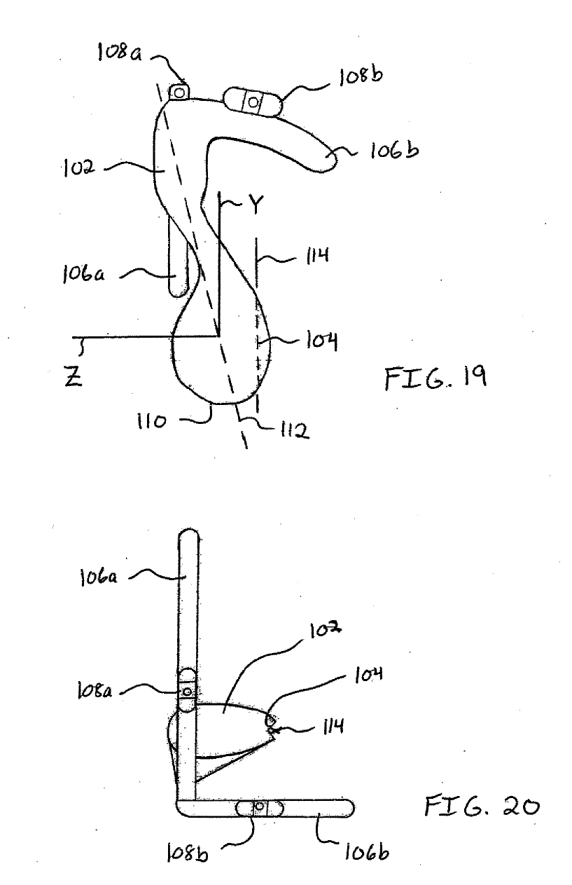

Referring to FIGS. 18-20, another exemplary medical implement positioning device 100 is shown. The positioning device 100 provides the operator with the ability to align a medical implement with a target axis by moving the implement in multiple planes. Occasionally, the optimal pathway or target axis for a CT or MR guided procedure is outside the standard imaging plane. This occurs when a normal or abnormal structure lying in the standard image plane must be avoided to gain access to the site of interest, such as a lesion. For example, a CT guided procedure might require a target pathway of 30 degrees in the X-Y plane and 20 degrees in the Y-Z plane. The X-Y plane may be the standard imaging plane, while the Y-Z plane may be a vertical plane perpendicular to the standard imaging plane.

The positioning device 100 includes a handle 102, an instrument guide 104, and a plurality of arcuate members and orientation indicators. The handle 102 may operate as a base of the positioning device 100 and may include a lower surface 110 which may be positioned adjacent or against a patient during use. The lower surface 110 may be configured for rotation on the patient's skin. For example, the lower surface 110 may be generally hemispherical. In use, an operator may contact the patient with the lower surface 110 and, optionally, simultaneously rotate the device 100 with respect to the patient. Thus, the positioning device 100 may include a lower surface 110 which may contact a patient and may be rotated by an operator with respect to the patient while the operator adjusts an orientation of the translation axis with respect to a desired target axis. Because the lower surface 110 of the handle 102 is hemispherical, the positioning device 100 is free to rotate about the lower surface 110 in multiple planes. When placed in contact with the patient, the lower surface 110 may sink into and be stabilized by the soft tissues with the distal portion of the instrument guide 104 positioned against but not significantly depressing the surface of the skin. This allows the device 100 to be stabilized without altering the calculated depth of penetration.

The handle 102 may include a major axis 112 which may be in the same plane as the translation axis 114 of the instrument guide 104. The handle 102 may be configured to allow the right or left band of an operator to freely and manually manipulate the device. The handle 102 may include markers, such as vertical markers, allowing a user to determine a distance which an instrument has translated with respect to the handle 102. The markers may be centimeter markers on the front and sides and may include a bold score at desired intervals (e.g., every 5 or 10 cm). The positioning device 100 may further include a vertical ruler, fixed or detachable with respect to the handle and parallel to the translation axis of an instrument operatively associated with the instrument guide. Also, a slide may be used to mark the distance of the desired penetration depth from the level of any reference point on the instrument.

As further seen in FIGS. 18-20, the instrument guide 104 preferably allows the operator to translate a medical implement along a translation axis 114 of the instrument guide 104. The instrument guide 104 may be of similar type, configuration, and dimension as previously described herein. For example, the instrument guide 104 may be a commercially available instrument guide, may be secured either permanently or releasably with respect to the handle, may be integrated in the handle or offset from the handle, may include a quick-release/quick-recapture configuration for the medical implement, and may be a simplified instrument guide with a V-shaped groove.

The positioning device 100 may further include one or more orientation indicators and arcuate members as previously described herein. In an exemplary embodiment, the device 100 includes two arcuate members 106a and 106b, each with an orientation indicator 108a and 108b. The arcuate members 106a and 106b are attached to the handle 102 and are positioned generally perpendicular to each other. The arcuate members 106a and 106b may be secured with respect to the handle 102 such that the zero degree calibration is parallel to the translation axis 114. Also, as previously described, the arcuate members 106a and 106b may be reversibly attached to the handle 102 so that the arcuate members 106a and 106b can be positioned in different orientations. Therefore, the device 100 can be held in either hand, used from either side of the table of the imaging system, and placed on the patient in different orientations.

The orientation indicators 106a and 106b may provide data indicative of angular deviations between the orientation indicators 106a and 106b and a reference axis, like the vertical axis. For example, the first arcuate member 106a and first orientation indicator 108a may be positioned on the handle 102 so that the first arcuate member 106a is generally parallel with the standard imaging plane (X-Y plane). The second arcuate member 106b and second orientation indicator 108b may be positioned generally perpendicular to the first arcuate member 106a and, therefore, generally perpendicular to the standard imaging plane (X-Y plane). When the positioning device 100 is rotated about the lower surface 110 of the handle 102 in the X-Y plane, the first orientation indicator 108a shows the deviation angle between the vertical axis (or Y axis) and the translation axis 114 of the instrument guide 104 in the X-Y plane. Likewise, when the positioning device 100 is rotated about the lower surface 110 in the Y-Z plane, the second orientation indicator 108b shows the deviation angle between the vertical axis (or Y axis) and the translation axis 114 of the instrument guide 104 in the Y-Z plane. When the first and second orientation indicators 108a and 108b show the desired deviation angles, the operator has aligned the translation axis 114 with the target axis.

The orientation indicators 108a and 108b may be of any type, dimension, and configuration as previously described herein. Preferably, each orientation indicator 108a and 108b includes a small transparent fluid-filled cylindrical chamber having a bubble therein. Such an orientation indicator allows the operator to see the bubble regardless of the angle at which the indicator is viewed. Such an orientation indicator also limits the deviation detection to a single angle. For example, the bubble in orientation indicator 108 a should not move when position device 100 is rotated about the lower surface 110 in the Y-Z plane. The orientation indicators 108a and 108b preferably slide along the arcuate members 106a and 106b that are calibrated and marked in degrees. In use, the operator slides the indicators 105a and 108b to the desired angle on the arcuate members 106a and 106b. The instrument guide 104 is loaded with the medical implement. The operator places the positioning device 100 on the patient with the tip of the medical implement on the skin puncture site and the bubbles of the orientation indicators 108a and 108b centered within the chambers. The positioning device 100 is now at the desired deviation angles in the X-Y and Y-Z planes.

It is contemplated that the medical implement positioning device 100 of FIGS. 18-20 may include features and components previously described herein. For example, the positioning device may include a vertical ruler, a ruler slide, guide markers, and handle markers.

It is further contemplated that a sterile sleeve may be utilized with the various embodiments of the positioning device described herein. For example, a sterile sleeve may be placed over the handle and the orientation indicator shown in FIGS. 1-3; then a conventional sterile instrument guide can be attached. A sterile sleeve (with or without a groove) may be placed over the handle with a groove shown in FIGS. 12 and 13 and the orientation indicator. A sterile sleeve can be placed over the handle with a groove and the orientation indicator; then a conventional instrument guide can be attached. Specifically, a conventional sterile instrument guide can be clipped, snapped, or fastened to a slightly modified base covered with a sterile sleeve.

It is also contemplated that in using any of the embodiments herein having a groove as a simplified instrument guide, if significant friction is created between a sterile glove on the operator's fingers and the implement (i.e. soft plastic drainage catheter), a sterile low friction plastic flap can be interposed between the operator's fingers and the implement, without loss of tactile control of position of the implement.

While the above invention has been described with reference to certain preferred embodiments, it should be kept in mind that the scope of the present invention is not limited to these. For example, sizes, angles, and other dimensions discussed in the text and shown in the figures are merely exemplary. Thus, one skilled in the art may find variations of these preferred embodiments which, nevertheless, fall within the spirit of the present invention, whose scope is defined by the claims set forth below.

What is claimed is:

1. A device for guiding a medical instrument with respect to a patient, the device comprising:
a base having upper and lower portions, the lower portion including a bottom surface having a configuration for contact with the patient;
an instrument guide attached to the lower portion of the base, the guide having a translation axis and configured to slideably receive the medical instrument along the translation axis;
first and second arcuate members attached to the base, the arcuate members being generally perpendicular to each other; and
first and second orientation indicators connected with the first and second arcuate members, respectively.

2. The device of claim 1, wherein the bottom surface is configured for rotation in multiple planes.

3. The device of claim 2, wherein the base includes a major axis intersecting the bottom surface and the base is configured to be received within a hand of an operator about the major axis.

4. The device of claim 3, wherein the base is a unitary injection molded handle.

5. The device of claim 1, wherein the instrument guide is removably attached to the base.

6. The device of claim 5, wherein the instrument guide is configured for quick release and recapture of the medical instrument.

7. The device of claim 6, wherein the instrument guide includes a V-shaped groove.

8. The device of claim 1, wherein the arcuate members are reversibly attached to the base.

9. The device of claim 8, wherein the orientation indicators are slideably attached to the arcuate members.

10. The device of claim 9, wherein each orientation indicator includes a transparent chamber having a bubble in liquid.

11. The device of claim 1, wherein the base includes an attachment point configured for connection to an attachment arm.

12. A method for guiding a medical instrument along a target axis within a patient using the positioning device of claim 1, the method comprising:
positioning the medical instrument in the instrument guide;
positioning the bottom surface of the base against the patient with the distal end of the medical instrument positioned near a puncture site of the patient;
rotating the positioning device about the bottom surface until the translation axis is generally aligned with the target axis; and
translating the medical instrument along the translation axis and through the puncture site of the patient.

13. The method of claim 12, further comprising determining a first deviation angle of the target axis in a first plane.

14. The method of claim 13, further comprising positioning the first orientation indicator on the first arcuate member at indicia representing the first deviation angle.

15. The method of claim 14, wherein positioning the bottom surface of the base includes positioning the first arcuate member generally parallel with the first plane.

16. The method of claim 15, wherein rotating the positioning device includes rotating the positioning device in the fist plane to center a bubble in the first orientation indicator.

17. The method of claim 16, further comprising determining a second deviation angle of the target axis in a second plane, the second plane being generally perpendicular to the first plane.

18. The method of claim 17, further comprising positioning the second orientation indicator on the second arcuate member at indicia representing the second deviation angle.

19. The method of claim 18, wherein positioning the bottom surface of the base includes positioning the second arcuate member generally parallel with the second plane.

20. The method of claim 19, wherein rotating the positioning device includes rotating the positioning device in the second plane to center a bubble in the second orientation indicator.
